(19) United States
(12) Patent Application Publication
(10) Pub. No.: US 2005/0131530 A1
(43) Pub. Date: Jun. 16, 2005

(54) Endoluminal stent

(76) Inventor: Ed Eugene Darack, Fort Garland, CO (US)

Correspondence Address: Ed Darack, P.O. Box 413, Fort Garland, CO, 81133, US

(21) Appl. No.: 10/737,070

(22) Filed: Dec. 15, 2003

(51) Int. Cl. A61F 2/06

(52) U.S. Cl. 623/1.16; 623/1.15

(57) ABSTRACT

An endoluminal stent for placement in a body lumen, particularly a human coronary artery. The stent consists of a plurality of expansion rings aligned about a common axis, the expansion rings being comprised of a plurality of substantially ovaloid support units interconnected by rotatable struts, the expansion rings interconnected by a plurality of substantially sinusoidal struts. The stent can be expanded to a great degree due both to the substantially ovaloid support units plastically deforming to a substantially circular shape and the interconnecting struts rotating from an alignment substantially parallel to the stent's longitudinal axis to an alignment substantially orthogonal to the stent's longitudinal axis during stent placement. The stent may be successfully placed within a tortuous body lumen due to the stent's sinusoidal support ring interconnect struts. The design minimizes the stent-body lumen contact area to promote rapid endothelialization and minimization of thrombogenicity, reducing the chance of restenosis.

### BACKGROUND OF THE INVENTION—FIELD OF INVENTION

This invention relates to minimally invasive endoluminal prosthetic devices intended to provide internal, radially expansile scaffolding of a body lumen, and more specifically, such devices intended to open and/or maintain the open state of occluded human coronary arteries in such a manner as to minimize coronary artery restenosis subsequent to stent placement and to allow for a high degree of radial stent expansion and stent articulation during stent placement.

### BACKGROUND OF THE INVENTION—DESCRIPTION OF PRIOR ART

Endoluminal stents, in particular, those intended for placement in human coronary arteries for the purpose of opening and/or maintaining the open state of coronary luminal passageways occluded by atheroscleratic legions, are well known in the art. Stents, as are currently known, are crimped onto deflated balloon catheters and inserted into the human vasculature and guided to the stenosed section of the coronary artery, where they are radially expanded and mechanically affixed via balloon inflation for permanent or semi-permanent endoluminal scaffolding to restore and/or maintain normal hemodynamic flow. While the current field of the art represents a substantial improvement over previous revascularization techniques, a number of problems persist. One of the most troubling problems is post stent-placement restenosis. Another problem with the current field of the art is a relatively low crimped-state to expanded-state ratio of the stent cross-sectional diameter. Yet another problem is lack of articulation along the longitudinal axis of stents for placement in tortous sections of occluded body lumens. No current stent design adequately addresses all of these problems.

U.S. Pat. No. 5,443,496 to Schwartz et al. (1995) allows for limited articulation along the longitudinal axis of the stent. U.S. Pat. No. 5,964,798 to Imran (1999) allows for limited radial expansion. U.S. Pat. Nos. 6,352,552 to Levinson et al. (2002), 6,607,554 to Dang et al. (2003), 6,613,081 to Kim et al. (2003), and 6,652,573 to von Oepen (2003) allow for limited articulation and utilize a continuous web structure that presents a higher than needed stent surface to body lumen contact area, which is a contributing factor in restenosis.

### BRIEF SUMMARY OF THE INVENTION

This invention provides an improved design for endoluminal stents, particularly those stents intended for human cornonary artery revascularization where the design should allow for a very small crimped diameter for guidance through or placement within highly occluded luminal passageways, a high degree of expansion to restore normal vascular patency at the legion situs, a high degree of articulation for guidance through or placement within tortuous sections of luminal passageways, and where the total stent surface area in contact with the luminal wall should be minimized as much as possible to reduce damage to the endothelium, the natural nonthrombogenic lining of the arterial lumen, at the stent situs during stent placement. It is generally known in the art that the relatively high restenosis rate for current stent designs results from a failure of these stents to become endothelialized subsequent to stent placement. Damage to the endothelium during stent placement exposes thrombogenic proteins, resulting in platelet aggregation and the formation of thrombi. Damage to the endothelium during stent placement is also known to promote neointimal hyperplasia. Accordingly, a stent design that minimizes the total stent area that makes contact with the luminal wall is considered critical in minimizing endothelial damage and post stent placement restenosis.

In a preferred embodiment, the stent of the present invention includes a plurality of distinct expansion rings aligned about a common longitudinal axis, where each expansion ring has a first, compressed diameter, and a second, expanded diameter. Each expansion ring includes a plurality of distinct support units that are in communication with one another by interconnect struts. In the stent's first, compressed diameter the support units are of a substantially oval shape. The interconnect struts communicate directly with the support units at locations substantially on the medial aspect of the support units. In the stent's first, compressed diameter, the interconnect struts are aligned substantially parallel to the longitudinal axis of the stent. Upon stent placement, the stent assumes its second, expanded diameter. In the stent's second, expanded diameter, the support units are of a substantially circular shape and the interconnect struts are of a substantially orthogonal alignment to the longitudinal axis of the stent. The expansion rings are in communication with one another by a plurality of sinusoidal struts. These sinusoidal struts connect the expansion rings to one another at each respective expansion ring's support units.

Objects and Advantages

Accordingly, besides the objects and advantages of the endoluminal stent described in my above patent, several objects and advantages of the present invention are:
(a) to provide an endoluminal stent that has a small first, compressed diameter;
(b) to provide an endoluminal stent that has a high degree of radial expansion;
(c) to provide an endoluminal stent that can be guided through tortuous sections of body lumens;
(d) to provide an endoluminal stent that minimizes endothelial damage during placement by minimizing the stent-lumen contact area.

My invention resides not in any one of these features, but rather in the particular combination of all of them herein disclosed and claimed and it is distinguished from the prior art in this particular combination of all its structures for the functions specified. Those skilled in the art will appreciate that the conception, upon which this disclosure is based, may readily be utilized as a basis for the designing of other structures, methods, and systems for carrying out the several purposes of the present invention. It is important, therefore, that the claims be regarded as including such equivalent constructions insofar as they do not depart from the spirit and scope of the present invention. Still further objects and advantages will become apparent from a consideration of the ensuing description and drawings.

### DRAWING FIGURES

FIG. 1 shows, in a two dimensional layout, the novel endoluminal stent of the present invention.

FIG. 2 shows, in a two dimensional layout, the expansion ring of the novel endoluminal stent of the present invention, in a first, compressed state.

FIG. 3 shows, in a two dimensional layout, the expansion ring of the novel endoluminal stent of the present invention, in a second, expanded state.

### REFERENCE NUMERALS IN DRAWINGS

10 support unit

20 rotatable strut

30 sinusoidal strut

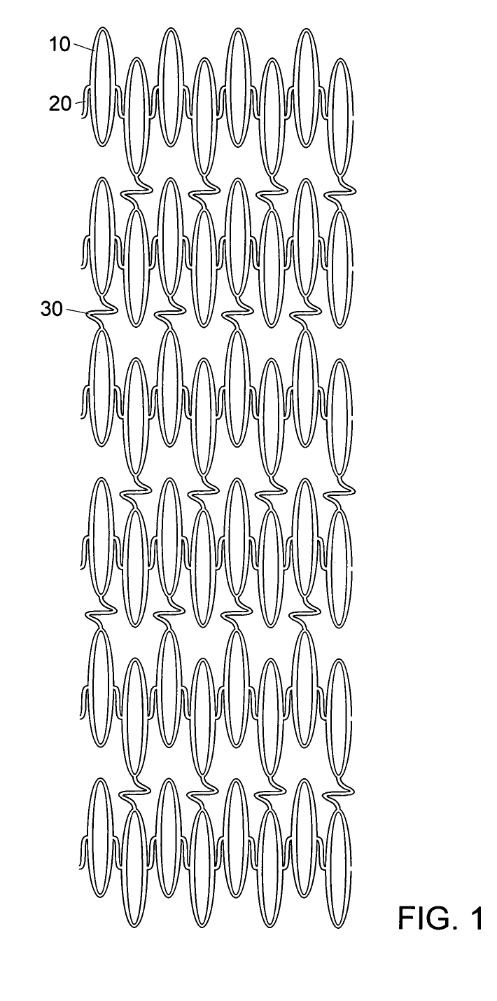

### DESCRIPTION—FIGS. 1 THROUGH 3—PREFERRED EMBODIMENT

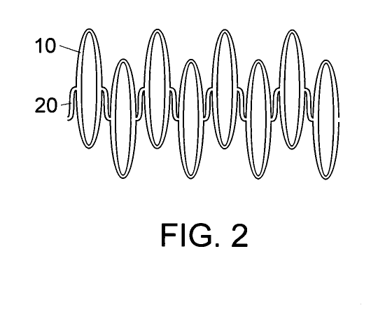

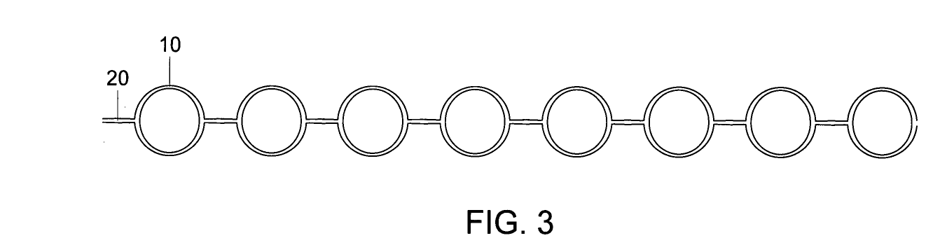

The preferred embodiment of the endoluminal stent of the present invention is illustrated in FIGS. 1, 2, and 3. FIG. 1 shows, in a two dimensional layout, a complete stent of the present invention. The stent is comprised of a plurality of expansion rings, each expansion ring being comprised of a plurality of support units 10 interconnected by rotatable struts 20. The expansion rings are interconnected by means of sinusoidal struts 30. FIG. 2 illustrates the expansion ring of the present invention in a first, compressed state; FIG. 3 illustrates the expansion ring of the present invention in second, expanded state. Due to the invention's unique and novel geometry, which utilizes expansion of both the support units 10 and rotation of the interconnecting rotatable struts 20, the endoluminal stent of the present invention is able to attain a very high degree of expansion. The endoluminal stent of the present invention utilizes discreet support units for the scaffolding of a body lumen as opposed to a continuous web design, reducing the overall stent-luminal wall contact area, resulting in lowered endothelial damage during stent placement and hence reducing the risk of restenosis subsequent to stent placement. The endoluminal stent of the present invention utilizes discreet support units for the scaffolding of a body lumen as opposed to a continuous web design, allowing for a highly compact first, compressed state.

In the first, compressed state of the endoluminal stent of the present invention, the support units 10 are arranged in a staggered geometry relative to one another, this staggering being geometrically constrained by the length of the rotatable struts 20 in the first, compressed state while the rotatable struts are aligned substantially parallel to the longitudinal axis of the endoluminal stent. The rotatable struts 20 communicate directly with the support units 10 at the substantially medial aspect of the outer perimeter of the support units. Upon expansion, the support units 10 widen to a substantially circular shape and the rotatable struts 20 rotate to a position substantially orthogonal to the longitudinal axis of the endoluminal stent.

The expansion rings are interconnected by the sinusoidal struts 30 by making direct communication between support units 10 of respective interconnected expansion rings. The sinusoidal struts 30 are positioned at regular, evenly spaced intervals between respective respective interconnected expansion rings, such that the sinusoidal struts 30 do not interfere with stent radial expansion and allow maximum articulation of the endoluminal stent in both compressed and expanded states.

The endoluminal stent of the present invention may be constructed of a number of materials and fashioned by a variety of means. The endoluminal stent of the present invention may be constructed of stainless steel, cobalt-based superalloys, titanium, tantalum, or other such appropriate metals. The endoluminal stent of the present invention may also be constructed of bioactive and biocompatable materials such as expanded polytetrafluoroethylene-based polymers and the like. The endoluminal stent of the present invention may be fashioned by laser etching, high-pressure water etching, chemical etching, mechanical cutting, and cold-stamping.

### CONCLUSION, RAMIFICATIONS, AND SCOPE

Accordingly, the reader will see that the endoluminal stent of the present invention provides for a superior level of coronary revascularization and permanent endoluminal scaffolding. Due to its geometry, the endoluminal stent of the present invention may be crimped onto a delivery catheter with a very small diameter, be expanded to a great degree, articulate through highly tortuous intraluminal passageways, and upon expansion present a minimal stent-luminal wall contact area.

1. An endoluminal stent, comprising:
a substantially cylindrical body having a longitudinal axis and open proximal and distal ends, said endoluminal stent having a plurality of expandable rings oriented about said longitudinal axis, said expandable rings having a first compressed diameter and a second expanded diameter, said expandable rings being comprised of a plurality of substantially ovaloid support units arranged circumferentially about said longitudinal axis and interconnected to one another by rotatable struts, said expandable rings being interconnected to one another by a plurality of sinusoidal struts.
